(19) United States
(12) Patent Application Publication
(10) Pub. No.: US 2019/0266597 A1
(43) Pub. Date: Aug. 29, 2019

(54) Healthcare Syndicate Electronic Token

(71) Applicant: Panaxea Life, Inc., Wilmington, DE (US)

(72) Inventor: Omar Mohtar, Torrance, CA (US)

(21) Appl. No.: 16/251,869

(22) Filed: Jan. 18, 2019

Related U.S. Application Data
(60) Provisional application No. 62/624,515, filed on Jan. 31, 2018.

(51) Int. Cl. G06Q 20/36; G06Q 20/40; G16H 10/60; H04L 9/06

(52) U.S. Cl. CPC G06Q 20/363; G06Q 20/3676; G06Q 20/401; H04L 2209/38; H04L 9/0637; H04L 2209/56; G16H 10/60

(57) ABSTRACT

Embodiments provide an improved healthcare system that includes a two-tiered healthcare cryptocurrency used to digitally execute healthcare payment transactions between consumers and healthcare provides. The embodiments are directed to systems and methods for executing the healthcare payment transactions using the two-tiered healthcare cryptocurrency. The systems and methods deposit, by a healthcare service application, a sum of healthcare cryptocurrency to the healthcare service account of the user. The systems and methods receive by the user from a healthcare provider, through a user interface to the healthcare service application, a payment request for a health related service in a specified amount of the healthcare cryptocurrency. The systems and methods transmit by the user to the healthcare provider, through the user interface, a confirmation of the payment request in the specified amount. The systems and methods transfer, by the healthcare service application, the specified amount from the healthcare service account of the user to the healthcare service account of the healthcare provider. The systems and methods record, by the healthcare service application, the given health related service, the healthcare provider, and the specified amount in the blockchain.

### RELATED APPLICATION(S)

This application claims the benefit of U.S. Provisional Application No. 62/624,515 filed on Jan. 31, 2018. The entire teachings of the above application(s) are incorporated herein by reference.

### BACKGROUND

Healthcare innovation has moved at a very slow pace and is in dire need for a technological revolution to meet the rising demands of consumers. Healthcare innovation is not slowed down by the talent or number of physicians nor of the quantity of medical supplies in the U.S., but by “outsiders” adding layers of revisions to make it more “accessible.” The constant negotiations on insurance claims taking place behind the scenes between insurance companies and healthcare providers delay medical care and increase medical costs. Further, the inadequate storage of medical data prevents authorized and beneficial access to the medical data.

The advent of decentralized transaction systems, such as bitcoin and smart contracts, has provided the Internet with a reliably secure protocol for recording ownership over digital value known as the blockchain. The blockchain is rooted in private keys and signatures that enable users to digitally preserve immutable historical records (blocks) of transactions in a common ledger linked and secured by cryptology. The common ledger structure of the blockchain makes the records easy to write, read, and to verify their accuracy (e.g., via services/agency formed particularly to perform such functions for a user, entity, or computer system), while difficult for a malicious actor to alter the content or order of the records. The block chain is built on the backs of thousands of peered servers and devised to be mathematically impenetrable. As long as a majority of participating peers act in support of the community, one cannot leverage enough computing power to edit records of the past and steal value.

### SUMMARY

Embodiments of the present invention are directed to improving healthcare systems based on the power of the blockchain. The embodiments provide a computer application that executes a two-tiered digital healthcare cryptocurrency structure to provide an improved healthcare system. The computer application further establishes relationships between consumers, healthcare providers, and health insurance companies and facilitates digital distribution of the healthcare cryptocurrency based on the established relationships. The computer application further facilitates the digital execution of healthcare payment transactions between the consumers, healthcare providers, and health insurance companies using the healthcare cryptocurrency, and records these transactions in the blockchain. By using blockchain technology, the embodiments provide layers of security, interaction, and transparency in real-time.

Embodiments of the present invention are directed to computer methods, systems, and program products for executing healthcare transactions. The computer program products comprise a non-transitory computer-readable storage medium having code instructions stored thereon, the storage medium operatively coupled to a processor to execute the computer method embodiments. The computer systems comprise a first computing device coupled to a first user, and the first computing device providing a first user interface to a healthcare service application. The computer systems also comprise a second computing device coupled to a healthcare provider, and the second computing device providing a second user interface to the healthcare service application. The computer systems may also comprise one or more computing devices coupled to health insurance companies, and the one or more computing devices also providing interfaces to the healthcare service application. The computer systems also comprise a healthcare service processor communicatively coupled to the computing devices and the blockchain, and implementing the healthcare service application.

The computer methods, systems, and program products register the user and the healthcare provider with the healthcare service application communicatively coupled to the blockchain. The registering creates a respective healthcare service account for each of the user and the healthcare provider with the healthcare service application. The computer methods, systems, and program products deposit, by the healthcare service application, a sum of healthcare cryptocurrency to the healthcare service account of the user. The computer methods, systems, and program products receive by the user from a healthcare provider, through the first user interface to the healthcare service application, a payment request for a given health related service in a specified amount of the healthcare cryptocurrency. The computer methods, systems, and program products then transmit by the user to the healthcare provider, through the user interface to the healthcare service application, a confirmation of the payment request in the specified amount. In response, the computer methods, systems, and program products transfer, by the healthcare service application, the specified amount from the healthcare service account of the user to the healthcare service account of the healthcare provider. The computer methods, systems, and program products record, by the healthcare service application, the given health related service, the healthcare provider, and the specified amount in the blockchain.

In some embodiments, the computer methods, systems, and program products configure the healthcare cryptocurrency into a two tier structure. In this two-tiered structure, a first tier of the healthcare cryptocurrency is purchased directly from the healthcare service application and exchangeable in two-way transactions among the user, healthcare provider, and one or more health insurance companies. The second tier of the healthcare cryptocurrency is exchangeable in one-way transactions limited to the following. The user may purchase, via the healthcare service application, the second tier healthcare cryptocurrency from one or more health insurance companies. The user may pay for a given health related service using the second tier healthcare cryptocurrency. The healthcare provider may distribute the second tier healthcare cryptocurrency to the one or more health insurance companies at an agreed cost. The computer methods, systems, and program products generate the first tier healthcare cryptocurrency into the second tier cryptocurrency based on certain conditions. These certain condition includes at least one of: (i) acquiring a certain amount of the first tier healthcare cryptocurrency in a healthcare service account, (ii) having the first tier healthcare currency in the healthcare service account for a particular time, and (iii) frequency of transactions using the first tier healthcare currency.

In some embodiments, the computer methods, systems, and program products register one or more health insurance companies with the healthcare service application. The registering creates a respective healthcare service account for each of the one or more health insurance companies. The computer methods, systems, and program products configure the healthcare service application to distribute healthcare cryptocurrency received in the healthcare service account of the healthcare provider to each of the one or more health insurance companies in particular proportions. The computer methods, systems, and program products distribute, by the healthcare service application, the specified amount transferred from the user's healthcare service account to the respective healthcare service accounts of the one or more healthcare insurance companies according to the particular proportions. In some of these embodiments, the computer methods, systems, and program products base the configured associated proportions for distributing the healthcare cryptocurrency on purchase agreements between the healthcare provider and the one or more health insurance companies.

In some embodiments, the computer methods, systems, and program products deposit the healthcare cryptocurrency in the user's account in response to the user transmitting, through the user interface to the healthcare service application, a request to purchase the sum of healthcare cryptocurrency. In some embodiments, the computer methods, systems, and program products deposit the healthcare cryptocurrency based on participation of the user in a subscription plan with a health insurance company as follows. The computer methods, systems, and program products enable the health insurance company to at least one of: (i) purchase, through an interface to the healthcare service application, a bulk amount of the healthcare cryptocurrency from the healthcare service application, and (ii) receive, through the interface to the healthcare service application, a distribution of the second tier healthcare cryptocurrency from one or more healthcare providers. The computer methods, systems, and program products further enable the health insurance company to store the purchased or received healthcare currency in a healthcare service account for the health insurance company. The computer methods, systems, and program products then automatically and periodically transfer, via the healthcare service application, a specific quantity of the bulk amount from the healthcare service account of the health insurance company to the healthcare service account of the user.

In example embodiments, an encrypted digital wallet is coupled to each healthcare service account for storing and processing the healthcare cryptocurrency. In example embodiments, smart contracts are executed by the healthcare service application to perform transferring of the healthcare cryptocurrency and recording in the blockchain. In some embodiments, a purchase of healthcare cryptocurrency is paid for using at least one of: other digital currency, U.S. currency, foreign government currency, and fiat currency.

Embodiments of the present invention are also directed to computer methods, systems, and program products for recording and searching healthcare transactions. The computer program products comprise a non-transitory computer-readable storage medium having code instructions stored thereon, the storage medium operatively coupled to a processor to execute the computer method embodiments. The computer systems comprise a computing device coupled to a healthcare provider, and the computing device providing a user interface to a healthcare service application. The computer systems also comprise a healthcare service processor communicatively coupled to the computing device and the blockchain, and implementing the healthcare service application.

The computer methods, systems, and program products register a user and a healthcare provider with a healthcare service application communicatively coupled to a blockchain. The registering creates a pair of a public key and a private key for each of the user and the healthcare provider. The computer methods, systems, and program products perform a dual login to the healthcare service application by providing the public key of the user and the public key of the healthcare provider. In example embodiments, the user only provides the private key of the user to the healthcare service application a first time the user associates with (e.g., visits) a new healthcare provider registered with the healthcare service application. The computer methods, systems, and program products then submit, through the healthcare service application, one or more medical transactions of the user with the healthcare provider. Each of the one or more medical transactions being assigned a token representing value (cost) of the medical transaction and at least one flag categorizing the medical transaction. In some embodiments, the one or more medical transactions include at least one of: a lab test, a medical procedure, a medical diagnosis, and another medical billing expense.

The computer methods, systems, and program products next generate a service packet that includes the one or more submitted medical transactions during the user's association (e.g., visit) with the healthcare provider. The service packet being stored in a private ledger of the healthcare service application. In some embodiments, a different private ledger is configured for each healthcare provider registered with the healthcare service application. The computer methods, systems, and program products update the generated service packet to include: (i) a cumulative token representing a total value of the tokens assigned to the one or more medical transactions in the service packet, (ii) a cumulative flag of the flags assigned to the one or more medical transaction in the service packet, and (iii) public keys of the user and healthcare provider. The computer methods, systems, and program products then create a public packet based on the generated service packet. The created public packet being stored in a public ledger of the healthcare service application. The public record includes: (i) a cost of service for the service packet in terms of a medical credit, (ii) unique flags that is a de-personalized and de-privatized compilation of the flags assigned to the one or more medical transaction in the service packet, and (iii) a secure identifier of the service packet. The computer methods, systems, and program products enable searching medical data of the public ledger based on the unique flag of each public packet stored in the public ledger.

In some embodiments, the computer methods, systems, and program products enable searching the public ledger by a searching healthcare provider registered with the healthcare service application as follows. The computer methods, systems, and program products locate the public record of the last service packet stored for the user in the public ledger using the public key of the user. The computer methods, systems, and program products then determine the unique flags of the public record to use as parameters for searching the public ledger. The computer methods, systems, and program products next search the public ledger to locate a subset of public records associated with the user and match the determined unique flags. The computer methods, systems, and program products use the service packet identifiers contained in the subset of public records to locate the service packets associated with the public records in the private ledger. The computer methods, systems, and program products return medical date of the located service packets to the registered searching healthcare provider.

In some of these embodiments, the searching healthcare provider is one or more of the following. A researcher that is locating the medical data of the user for performing a research study by comparing the medical data of the user to initial medical data of the study. A health insurance company locating the public record for performing auditing by using the cost of medical services from the cumulative token to verify medical expenses imposed on the user. A product company locating the public record for performing clinical trials by tracking products in the clinical trials using the cumulative flags of the service packet.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A is an example digital processing environment in which embodiments of the present invention may be implemented.

FIG. 1B is a block diagram of any internal structure of a computer/computing node.

FIG. 2A is an example system and method for distributing healthcare digital cryptocurrency in embodiments of the present invention.

FIG. 2B is an example system and method for forming a healthcare syndicate to distribute healthcare digital cryptocurrency in embodiments of the present invention.

FIG. 2C is an example system and method for managing healthcare digital cryptocurrency in embodiments of the present invention.

FIG. 3A is an example system and method for performing healthcare transactions using healthcare cryptocurrency in embodiments of the present invention.

FIG. 3B is another example system and method for performing healthcare transactions using healthcare cryptocurrency in embodiments of the present invention.

FIG. 4A is an example system and method for storing medical data in embodiments of the present invention.

FIG. 4B is an example system and method for searching medical data in embodiments of the present invention.

### DETAILED DESCRIPTION

A description of example embodiments follows.

Current healthcare is burdened by the excessive administrative and overhead costs, confusing health policy plans with rising premiums, and the lack of public pricing information. Modernizing healthcare through blockchain technologies can cut these expenses, accelerate the negotiation process, cut down on delays, and reduce fraudulent pricing. Embodiments of the present invention provide a way to obtain medical care with none of the middlemen or excessive costs, and to handle all medical claim transactions between health insurance companies (HIC) and health insurance providers (HCP) in an instant and transparent manner.

Embodiments provide a computer application that executes a two-tiered digital healthcare cryptocurrency structure which provides an improved, decentralized healthcare system not bound by health insurance companies. The computer application establishes relationships between consumers, healthcare providers, and health insurance companies and facilitates digital distribution of the two-tiers of healthcare cryptocurrency based on the established relationships. The computer application further provides transparency in medical payment transactions between health insurance companies (HIC) and healthcare providers (HCP) and between consumers and HPCs to purchase health related (healthcare) services.

Some embodiments of the present invention are further described in the White Paper, “Panaxea: The Cure to Healthcare,” by Omar Mohtar, dated November 2017, which is included as Appendix A. This White Paper is herein incorporated by reference in its entirety

### Digital Processing Environment

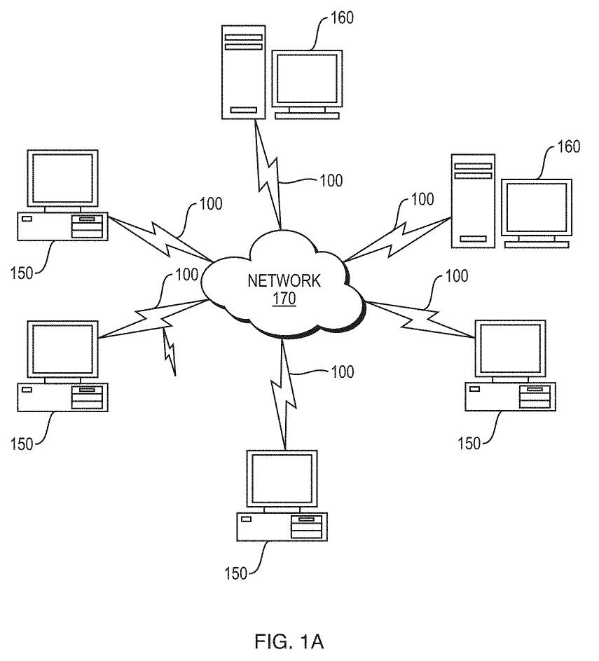

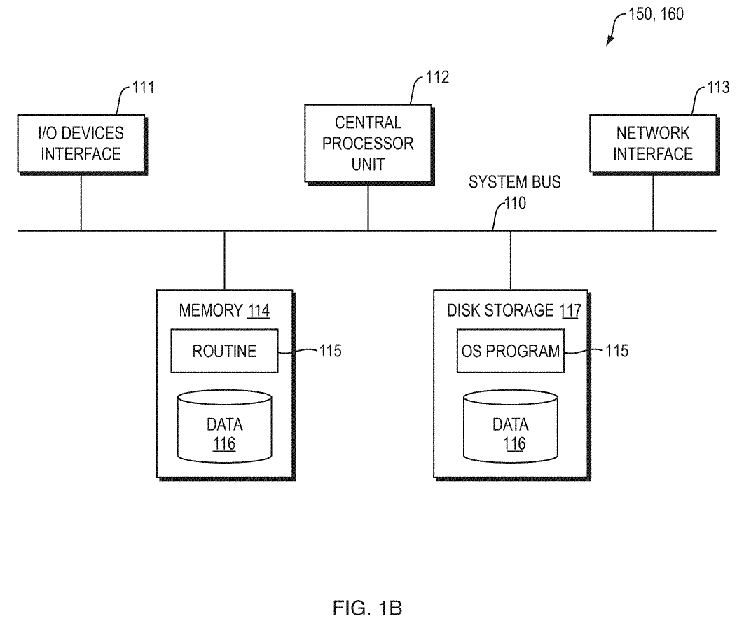

An example implementation of a healthcare service system 100 for executing and recording healthcare payment transactions according to embodiments of the invention may be implemented in a software, firmware, or hardware environment. The healthcare service system 100 includes a two-tier cryptocurrency structure for distributing and managing digital cryptocurrency used in the healthcare payment transactions. FIG. 1A illustrates one such example digital processing environment in which embodiments of the present invention may be implemented. Client computers/devices 150 and server computers/devices 160 provide processing, storage, and input/output devices executing application programs and the like.

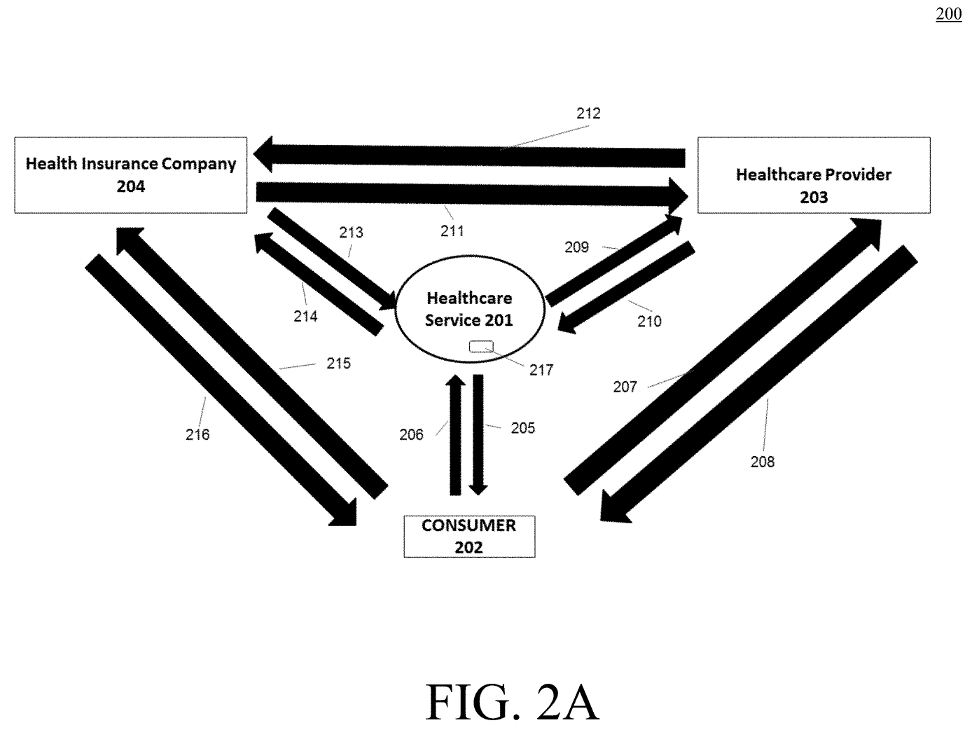

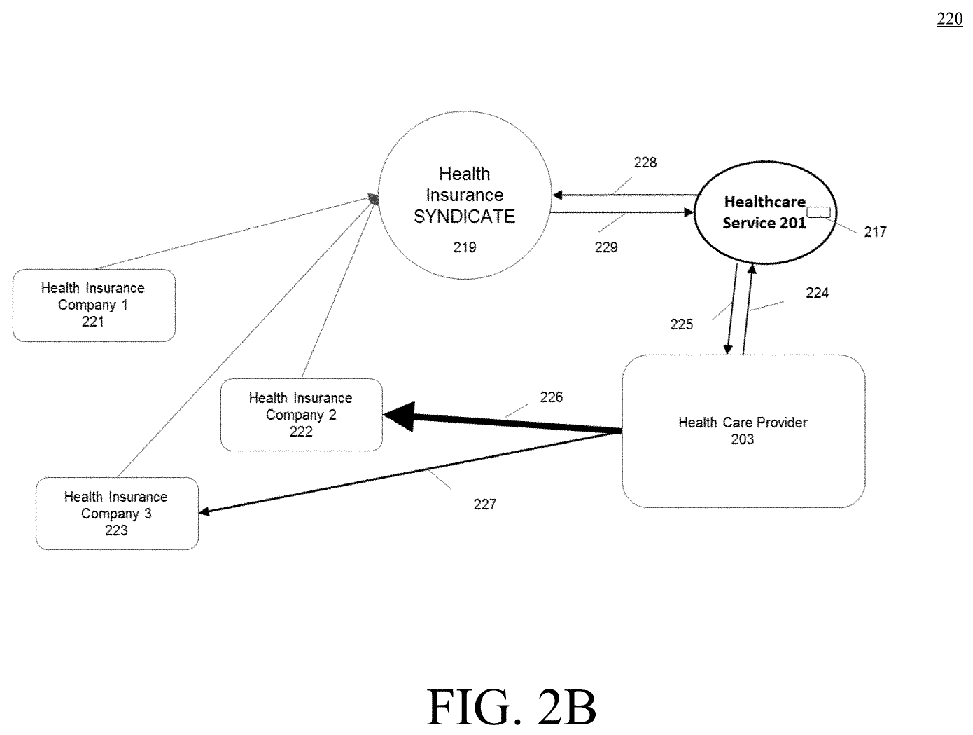

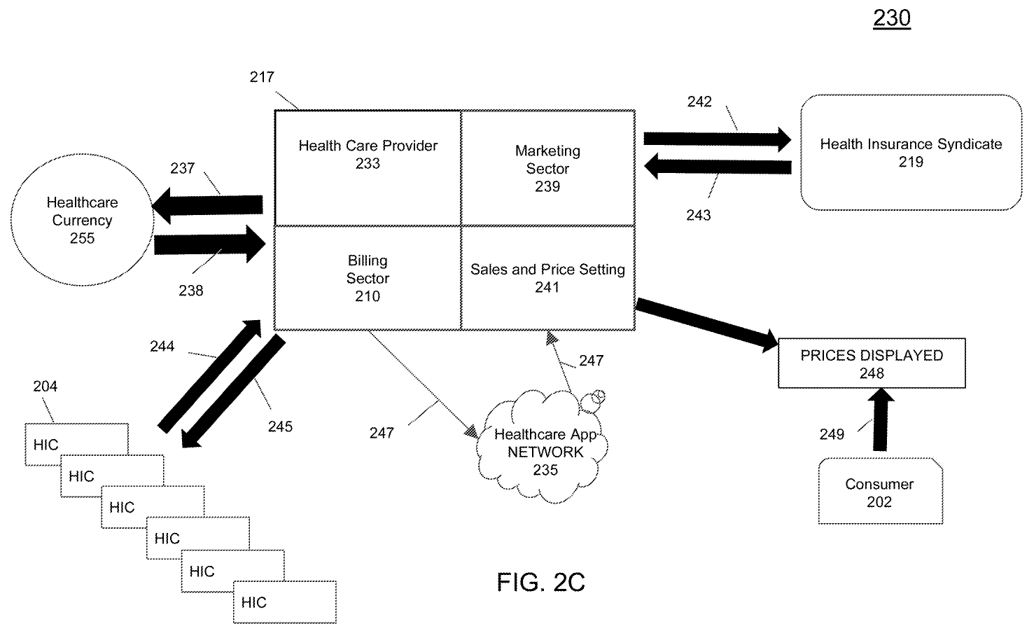

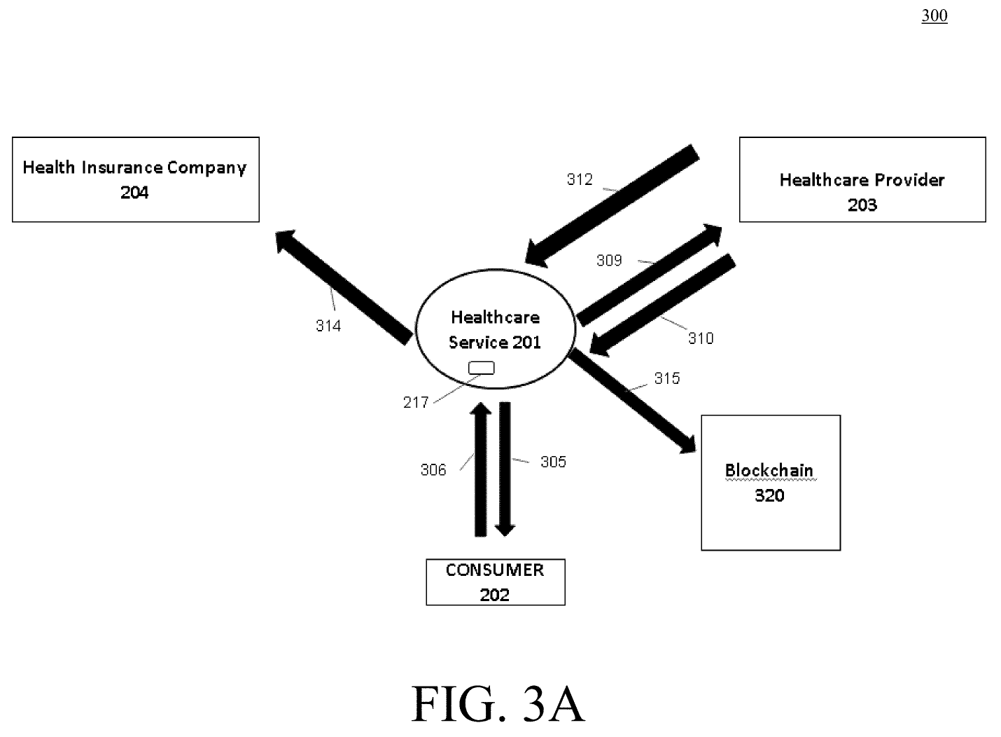

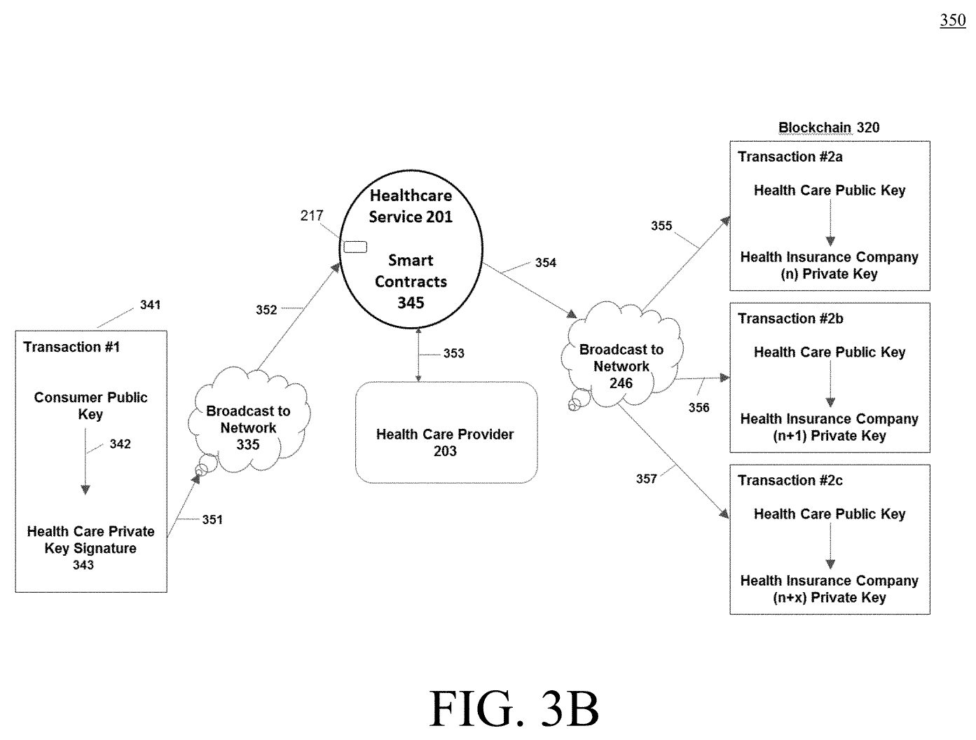

Client computers/devices 150 are linked through communications network 170 to other computing devices, including other client computers/devices 150 and server computer(s) 160. The network 170 can be part of a remote access network, a global network (e.g., the Internet), a worldwide collection of computers, Local area or Wide area networks, and gateways that currently use respective protocols (TCP/IP, Bluetooth, etc.) to communicate with one another. Other electronic device/computer network architectures are suitable. For example, client computers/devices 150 may include user/consumer devices 202 shown in FIGS. 2A-3B, which provide user interfaces to a healthcare service application 217. The client computers/devices 150 enable a user to communicate with the healthcare service application 217 to distribute and manage healthcare cryptocurrency and execute and record payment transactions.

Server computers 160 of the healthcare service system 100 may be configured to include the server 201 of FIGS. 2A-3B that executes the healthcare service application 217, which generates, distributes, and manages digital healthcare cryptocurrency purchased and exchanged between consumers, healthcare providers, and healthcare companies (as shown in FIGS. 2A-2C). The server 201 (via the healthcare service application 217) also executes and records in the blockchain healthcare payment transactions between a user and a healthcare provider (as shown in FIGS. 3A-3B). The Server computers 160 may be configured to further include a healthcare provider server 203 of FIGS. 2A-3B, which communication (via the healthcare service application 217) with consumers to acquire payment of healthcare cryptocurrency for healthcare services and with healthcare insurance companies to sell acquired healthcare cryptocurrency. The Server computers 160 may be configured to also include a health insurance company server 204 of FIGS. 2A, 26, and 3A, health insurance company servers 221-223 of FIG. 2B, and/or syndicate server 219 of FIGS. 2B-2C, which communicate (via the healthcare service application 217) with a consumer to sell acquired healthcare cryptocurrency or with a healthcare provider (or directly with the healthcare service application 217) to purchase healthcare cryptocurrency.

FIG. 1B is a block diagram of any internal structure of a computer/computing node (e.g., client processor/device/mobile phone device/tablet/video camera 150 or server computers 160) in the processing environment of FIG. 1A. Embodiments of the invention may include means for displaying audio, image, video or data signal information. Each computer 150, 160 in FIG. 1B contains a system bus 110, where a bus is a set of actual or virtual hardware lines used for data transfer among the components of a computer or processing system. Bus 110 is essentially a shared conduit that connects different elements of a computer system (e.g., processor, disk storage, memory, input/output ports, etc.) that enables the transfer of data between the elements.

Attached to system bus 110 is I/O device interface 111 for connecting various input and output devices (e.g., keyboard, mouse, touch screen interface, displays, printers, speakers, audio inputs and outputs, video inputs and outputs, microphone jacks, etc.) to the computer 150, 160. Network interface 113 allows the computer to connect to various other devices attached to a network (for example the network illustrated at 170 of FIG. 1A). Memory 114 provides volatile storage for computer software instructions 115 and data 116 used to implement software implementations of components of the present inventions (e.g. healthcare service systems 200, 220, 230, 300, 350 of FIGS. 2A-3C).

Software components 114, 115 of the healthcare service system 100 described herein may be configured using any known programming language, including any high-level, object-oriented programming language. The system 100 may include instances of processes that enable acquiring/purchasing healthcare cryptocurrency, selling healthcare cryptocurrency, transmitting healthcare payment transactions, and confirming and recording healthcare payment transactions. The healthcare service system 100 may also include instances of a trust scoring engine, which can be implemented as a client that communicates to the server 160 through SSL and computes a trust score that provides a measure of confidence that a transaction is trusted based on, for example, type of user (e.g., consumer, health service provider, health insurance company, and such) involved in the transaction, the amount of healthcare cryptocurrency used in the transaction, whether using a tier-one or tier-two type of healthcare cryptocurrency, the date/time of the transaction, and such.

In an example mobile implementation, a mobile agent implementation of the invention may be provided. A client-server environment can be used to enable mobile healthcare services using a healthcare network server. It can use, for example, the XMPP protocol to tether a healthcare network agent 115 on the device 150 to a server 160. The server 160 can then issue commands to the mobile device on request. The mobile user interface framework to access certain components of the system 100 may be based on XHP, Javelin, or WURFL. In another example mobile implementation for OS X and iOS operating systems and their respective APIs, Cocoa and Cocoa Touch may be used to implement the client side components 115 using Objective-C or any other high-level programming language that adds Smalltalk-style messaging to the C programming language.

Disk storage 117 provides non-volatile storage for computer software instructions 115 (equivalently “OS program”) and data 116 used to implement embodiments of the system 100. Central processor unit 112 is also attached to system bus 110 and provides for the execution of computer instructions.

In one embodiment, the processor routines 115 and data 116 are computer program products, e.g. healthcare service application 217, smart contracts 345, and trust scoring engine (generally referenced 115), including a computer readable medium capable of being stored on a storage device 117, which provides at least a portion of the software instructions for the healthcare services system 100. Executing instances of respective software components of the healthcare services system 100, such as instances of healthcare services application, smart contracts 345, and trust scoring engine may be implemented as computer program products 115, and can be installed by any suitable software installation procedure, as is well known in the art. In another embodiment, at least a portion of the system software instructions 115 may also be downloaded over a cable, communication and/or wireless connection via, for example, a browser SSL session or through an app (whether executed from a mobile or other computing device). In other embodiments, the system 100 software components 115 may be implemented as a computer program propagated signal product embodied on a propagated signal on a propagation medium (e.g., a radio wave, an infrared wave, a laser wave, a sound wave, or an electrical wave propagated over a global network such as the Internet, or other network(s)). Such carrier medium or signals provide at least a portion of the software instructions for the present healthcare services system 100.

### Method/System for Distributing Healthcare Cryptocurrency

FIG. 2A is an example method and system 200 for distributing digital healthcare cryptocurrency in embodiments of the present invention. The system 200 of FIG. 2A includes a healthcare service server (healthcare service processor) 201 that executes a healthcare service application 217 to distribute and manage the digital healthcare cryptocurrency based on a two-tier structure. The system 200 further includes a consumer (user) communicatively coupled with the healthcare service server 201 via a consumer device 202 (e.g., mobile phone, tablet, personal computer, and such). The consumer is a registered user having an account with the healthcare service application 217, and an encrypted digital wallet is coupled to the consumer's account for storing and processing the healthcare cryptocurrency. The consumer communicates with the healthcare service application 217 through a user interface on the consumer device 202 to the healthcare service application 217. The depicted consumer is representative of all non-health insurance and non-healthcare providers who use the digital currency for healthcare (medical) services.

The system 200 further includes a healthcare provider communicatively coupled with the healthcare service server 201 via a device 203 (e.g., mobile phone, tablet, personal computer, and such) in the network of the healthcare provider. The healthcare provider is also a registered user having an account with the healthcare service application 217, and an encrypted digital wallet is coupled to the healthcare provider's account for storing and processing the healthcare cryptocurrency. The healthcare provider communicates with the healthcare service application 217 through a user interface on the healthcare provider device 203 to the healthcare service application 217. The depicted healthcare provider is representative of all medical establishments such as clinics, hospitals, private practices, and such.

The system 200 further includes a health insurance company communicatively coupled with the healthcare service server 201 via a device 204 (e.g., mobile phone, tablet, personal computer, and such) in the network of the health insurance company. The health insurance company is also a registered user having an account with the healthcare service application 217, and an encrypted digital wallet is coupled to the health insurance company's account for storing and processing the healthcare cryptocurrency. The health insurance company communicates with the healthcare service application 217 through a user interface on the health insurance company device 204 to the healthcare service application 217. The depicted healthcare provider is representative of all health insurance companies that are involved currently with managing health care expenses and premium charging for consumers as well as working through healthcare providers.

The healthcare service application 217 manages the digital distribution of digital healthcare cryptocurrency between the consumer (via the consumer device 202), healthcare provider (via the healthcare provider device 203), and healthcare company (via the healthcare company device 204). To utilize such digital currency representing a healthcare network, through a healthcare service application 217, requires heavy modification and regulations in order to prevent improper and purely profit driven actions to persist. Stagnating the velocity of the digital healthcare cryptocurrency (also referred to in example embodiments as “Panaxea”) can drive up and down costs too quickly possibly reducing the buying power for consumers. This digital healthcare cryptocurrency is at its core for healthcare and providing transparent pricing. To prevent improper uses of this digital healthcare cryptocurrency, the healthcare service application 217 distributes and manages the digital healthcare cryptocurrency based on a two-tier token structure. The healthcare service application 217 only allows the second tier tokens to be used to purchase healthcare service.

The first tier tokens of the healthcare cryptocurrency may be purchased directly from the healthcare service (via the healthcare service application 217) by the consumer (via the consumer device 202), the healthcare provider (via the consumer device 203), and the health insurance company (via the consumer device 204), and is freely exchangeable (via the healthcare service application 217) in two-way transactions among the consumer (via the consumer device 202), the healthcare provider (via the consumer device 203), and the health insurance company (via the consumer device 204). As shown in FIG. 2A, the consumer via a user interface on the consumer device 202 to the healthcare service application 217 may directly purchase 205 (e.g., using other digital currency, U.S. currency, foreign government currency, fiat currency, and such) the first tier tokens directly from the healthcare service application 217 or other digital exchange, which is placed in the encrypted digital wallet coupled to the consumer's account. The consumer via a user interface on the consumer device 202 to the healthcare service application 217 may also purchase 205 the first tier tokens from other consumers, healthcare providers, and health insurance companies registered with the healthcare service application 217. The consumer via a user interface on the consumer device 202 to the healthcare service application 217 may also directly sell 206 (e.g., for other digital currency, U.S. currency, foreign government currency, fiat currency, and such) the first tier tokens to other consumers, service providers, and health insurance companies registered with the healthcare service application 217. Based on these transactions, the healthcare service application 217 transfers the first tier tokens to the encrypted digital wallet of the purchaser from the encrypted digital wallet of the seller.

As shown in FIG. 2A, the healthcare provider may via a user interface on the healthcare provider device 203 to the healthcare service application 217 directly purchase 209 (e.g., using other digital currency, U.S. currency, foreign government currency, fiat currency, and such) the first tier tokens directly from the healthcare service application 217 or other exchange, which is placed in the encrypted digital wallet coupled to the healthcare provider's account. The healthcare provider may also receive grants or other donations in the first tier currency, which is placed in the encrypted digital wallet coupled to the healthcare provider's account. The healthcare provider via a user interface on the healthcare provider device 203 to the healthcare service application 217 may also purchase 209 the first tier tokens from other healthcare providers, consumers, and health insurance companies registered with the healthcare service application 217. The healthcare provider via a user interface on the healthcare provider device 203 to the healthcare service application 217 may directly sell 210 (e.g., for other digital currency, U.S. currency, foreign government currency, fiat currency, and such) the first tier tokens to other service providers, consumers, and health insurance companies registered with the healthcare service application 217. Based on these transactions, the healthcare service application 217 transfers the first tier tokens to the encrypted digital wallet of the purchaser from the encrypted digital wallet of the seller.

As shown in FIG. 2A, the health insurance company may via a user interface on the health insurance company device 204 to the healthcare service application 217 directly purchase 214 (e.g., using other digital currency, U.S. currency, foreign government currency, fiat currency, and such) the first tier tokens directly from the healthcare service application 217 or other exchange, which is placed in the encrypted digital wallet coupled to the consumer's account. The health insurance company may purchase the first tier tokens for investing or loaning opportunities. The health insurance company via a user interface on the health insurance company device 204 to the healthcare service application 217 may also purchase 214 the first tier tokens from other health insurance companies, consumers, and healthcare providers registered with the healthcare service application 217. The health insurance company via a user interface on the health insurance company device 204 to the healthcare service application 213 may directly sell 213 (e.g., for other digital currency, U.S. currency, foreign government currency, fiat currency, and such) the first tier tokens to other health insurance companies, consumers, and healthcare providers registered with the healthcare service application 217. Based on these transactions, the healthcare service application 217 transfers the first tier tokens to the encrypted digital wallet of the purchaser from the encrypted digital wallet of the seller.

The first tier healthcare cryptocurrency stored in an encrypted digital wallet coupled to the account of a consumer, healthcare provider, or health insurance company may be generated into the second tier token based on certain conditions. The certain conditions include: (i) acquiring a certain amount of the first tier tokens in the encrypted digital wallet, (ii) having the first tier tokens in the encrypted digital wallet for a particular time, (iii) frequency of two-way transactions using the first tier tokens, and such.

The second tier tokens (referred to in some embodiments as “MedCoins”), may only be exchanged via the healthcare service application 217 in limited one-way transactions. First, the consumer via a user interface on the health insurance company device 202 to the healthcare service application 217 may purchase 207 (engage in a payment transaction for) a health related service from the healthcare provider using the second tier tokens, which are placed in the encrypted digital wallet of the consumer. The healthcare provider may then broadcast 208 via a user interface on the healthcare provider device 203 for all consumers registered with the healthcare service application 217 to view (e.g., in the blockchain). Second, the health insurance company may negotiate 211 with the healthcare provider for an exclusive contract (agreement) to buy a particular proportion or percentage of the second tier tokens acquired by the healthcare provider at a set cost. The healthcare provider via a user interface on the healthcare provider device 203 to the healthcare service application 217 (executing corresponding smart contracts) may distribute (sell) 212 the particular proportion of received second tier tokens to the health insurance company (via the health insurance company device 204) at the agreed cost, which are periodically placed in the encrypted digital wallet of the health insurance company. The healthcare service application 217 may be implemented (configured) to enforce the distribution at the agreed proportions and costs.

Third, the consumer via a user interface on the health insurance company device 202 to the healthcare service application 217 may purchase 215 the second tier tokens from the health insurance company. In some embodiments, the consumer may alternatively or additionally subscribe 216 to a plan with the health insurance company to obtain the second tier tokens (or in some embodiments first tier tokens) at a fixed interval and price from the health insurance company. In these embodiments, the health insurance company via a user interface on the health insurance company device 204 to the healthcare service application 217 may purchase a bulk amount of the first tier tokens from the healthcare service application. In these embodiments, the health insurance company via a user interface on the health insurance company device 204 to the healthcare service application 217 may also receive 212 distribution of second tier tokens from the healthcare provider based on an exclusive agreement with the healthcare provider. In these embodiments, the healthcare service application 217 stores the purchased or received second tier tokens in the encrypted digital wallet for the health insurance company. The healthcare service application 217 then periodically transfers a specific quantity of second tier tokens from the encrypted digital wallet of the health insurance company to the encrypted digital wallet of the consumer.

Note that these one-way transactions do not function in the reverse order. Further, the first tier and second tier tokens may be segregated in the encrypted digital wallet of the consumer, healthcare provider, and health insurance company by having an individual platform for each tier token.

### Method/System for Forming a Healthcare Syndicate

FIG. 2B is an example method and system 220 for forming a health insurance syndicate 219 to distribute healthcare digital cryptocurrency in embodiments of the present invention. The system 200 facilitates the forming and managing of a health insurance syndicate 219 that represents all health insurance companies (e.g., health insurance company 1 221, health insurance company 2 222, and health insurance company 3 223 of FIG. 2B) that agree to back the healthcare digital cryptocurrency. The health insurance companies 221, 222, 223 in the health insurance syndicate 219 agree to share a percentage of risk from all healthcare expenditures, such that as the health insurance syndicate 219 grows, the larger the coverage becomes for all consumers. The healthcare provider using the healthcare cryptocurrency agrees to only associate with health insurance companies within the health insurance syndicate 219.

The healthcare provider (via healthcare provider device 203) may negotiate 224 (via the healthcare service application 217 executing on the healthcare service server 201) with the health insurance companies 221, 222, 223 in the health insurance syndicate 219 to sell healthcare cryptocurrency that the healthcare provider will acquire for healthcare services. The interested health insurance companies 221, 222, 223 in the health insurance syndicate 219 (via respective computing devices) offer 225 to buy a set percentage (or proportion) of all the healthcare cryptocurrency acquired by the healthcare provider at a set price and for a specific duration of time.

In FIG. 2B, health insurance company 2 222 (via a user interface to the healthcare service application 217) offers 226 to buy a large percentage (e.g., 60%) of healthcare cryptocurrency to be acquired by the healthcare provider. The healthcare provider (via a user interface on healthcare provider device 203 to the healthcare service application 217) accepts the offer 226, and the healthcare service application 217 records the accepted terms (e.g., percentage, agreed cost, agreed duration, and such) for enforcing future transactions via smart contracts. In FIG. 2B, health insurance company 3 223 (via a user interface to the healthcare service application 217) offers 227 to buy a smaller percentage (e.g., 40%) of healthcare cryptocurrency to be acquired by the healthcare provider. The healthcare provider (via a user interface on healthcare provider device 203 to the healthcare service application 217) accepts the offer 227, and the healthcare service application 217 records the accepted terms (e.g., percentage, agreed cost, agreed duration, and such) for enforcing future transactions via smart contracts. In FIG. 2B, health insurance company 1 221 makes no offer to pay healthcare cryptocurrency to be acquired by the healthcare provider.

### Method/System for Managing Healthcare Cryptocurrency

FIG. 2C is an example method and system 230 for managing healthcare digital cryptocurrency in embodiments of the present invention. The system 230 includes the healthcare service application 217 comprising digital components (modules) configured to implement functions related to the healthcare provider 233, marketing sector 239, billing sector 210, and sales and pricing setting 241. A healthcare provider (via a healthcare provider device 203) interfaces through the healthcare provider management module 233 to request 238 registration with the healthcare service application 217 for usage of the healthcare digital cryptocurrency 255. The healthcare provider management module 233 responds 237 to approve the healthcare provider's request. The healthcare provider management module 233 may then setup an account (coupled to an encrypted digital wallet) for the healthcare provider.

The marketing sector module 239 of the healthcare service application 217 executes negotiation of contracts with health insurance companies to sell the healthcare digital cryptocurrency 255. The marketing sector module 239 of the healthcare service application 217 executes offers 242 to form a contract to sell a specified amount of healthcare digital cryptocurrency 255 to a syndicate 219 of health insurance companies 244 for a specified price (e.g., in other digital currency, U.S. currency, foreign government currency, fiat currency, and such) and duration. The health insurance syndicate 219 agrees 243 to the offers 243 by the marketing sector module 239.

In response to the agreement 243, the marketing sector module 239 communicates with the billing sector module 210, which executes divisions of healthcare cryptocurrency via smart contracts. The billing sector module 210 (via smart contracts) periodically (based on the agreement) divides the specified amount of the healthcare cryptocurrency between the health insurance companies 244. The billing sector module 210 (via smart contracts) then transfers 245 the divided healthcare cryptocurrency to the digital wallets of the respective health insurance companies 244 and collects the specified price from the health insurance companies 244. When the agreement expires, the billing sector module 210 (via smart contracts) communicates with the health insurance companies 244 (as the syndicate 219) to continue or adjust their contract for purchasing the healthcare cryptocurrency 255. The billing sector module 210 further broadcasts 247 all transfers (transactions) of healthcare cryptocurrency 255 on the healthcare service application network 235 to be seen by all consumer, healthcare providers, and health insurance companies on the network 235 (and may record the transactions in the blockchain).

The sales and pricing sector module 241 of the healthcare service application 217 periodically analyzes all regional pricing data for healthcare services to determine competitive prices to offer the healthcare cryptocurrency 255. The sales and pricing sector module 241 displays 248 the determined price to the consumers via a user interfaces to the consumer devices of the consumers or other digital interaction. A consumer 202 interested in purchasing healthcare services at the determined prices may communicate (via a user interface on the computer computing device 202 to the healthcare service application 217) to execute a purchase transaction 249 of an amount of healthcare cryptocurrency 255 at the determined price. Upon completion of the purchase, the sales and pricing sector module 241 puts the purchased amount of healthcare cryptocurrency 255 in the encrypted digital wallet couple to the consumer's healthcare service account.

### Method/System for Performing Healthcare Transactions

FIG. 3A is an example method and system 300 for performing healthcare transactions using healthcare cryptocurrency in embodiments of the present invention. The system 300 of FIG. 3A includes a healthcare service server (healthcare service processor) 201 that executes a healthcare service application 217 to perform payment transactions for healthcare services using the digital healthcare cryptocurrency described above in reference to FIG. 2A. The system 300 further includes a registered consumer (user) of the healthcare service application 217 that is communicatively coupled with the healthcare service server 201 via a consumer device 202 (e.g., mobile phone, tablet, personal computer, and such). The healthcare service account of the registered consumer is coupled to an encrypted digital wallet, and contains an amount of digital healthcare cryptocurrency (e.g., second tier tokens) acquired or deposited through methods described above in reference to FIG. 2A. The registered consumer communicates with the healthcare service application 217 through a user interface on the consumer device 202 to the healthcare service application 217.

The system 300 further includes a registered healthcare provider of the healthcare service application 217 that is communicatively coupled with the healthcare service server 201 via a healthcare provider device 203 (e.g., mobile phone, tablet, personal computer, and such). The healthcare service account of the registered healthcare provider is coupled to an encrypted digital wallet, and may contain an amount of digital healthcare cryptocurrency acquired through methods described above in reference to FIG. 2A. The registered healthcare provider communicates with the healthcare service application 217 through a user interface on the healthcare provider device 203 to the healthcare service application 217.

The system 300 further includes a registered health insurance company of the healthcare service application 217 that is communicatively coupled with the healthcare service server 201 via a health insurance company device 204 (e.g., mobile phone, tablet, personal computer, and such). The healthcare service account of the registered health insurance company is coupled to an encrypted digital wallet, and may contain an amount of digital healthcare cryptocurrency acquired through methods described above in reference to FIG. 2A. The registered health insurance company communicates with the healthcare service application 217 through a user interface on the healthcare provider device 203 to the healthcare service application 217.

The registered consumer via a user interface on the consumer device 202 to the healthcare service application 217 consults a published list of published healthcare cryptocurrency prices for healthcare services within the consumer's area. The healthcare service application 217 may generate the list based on payment transactions (in healthcare cryptocurrency) recorded on the blockchain for healthcare services previously performed in the consumer's area. The registered consumer has a particular healthcare service performed by a registered healthcare provider of the healthcare service application 217 based on the published list of healthcare cryptocurrency prices for healthcare services.

The registered healthcare provider via a user interface on the healthcare provider device 203 to the healthcare service application 217 sends 310 a payment request for the particular healthcare service in a specified amount of the healthcare cryptocurrency. The healthcare service application 217 receives the payment request and performs any necessary validation of the health service account of the healthcare provider (e.g., valid registration with the healthcare service application 217 and such). The healthcare service application 217 forwards 305 the payment request via a user interface on the consumer device 202 to the consumer. The consumer reviews the payment transaction to confirm the indicated healthcare service and specified payment amount are in accordance with a published list and/or agreements previously reached by the consumer and healthcare provider. If so, the consumer via the user interface on the consumer device 202 to the healthcare service application 217 sends 306 a payment confirmation of the particular healthcare service in the specified amount of the healthcare cryptocurrency.

The healthcare service application 217 receives the payment request and performs any necessary validation of the consumer of the health service account of the consumer (e.g. sufficient amount of healthcare cryptocurrency in the consumer's digital wallet). The healthcare service application 217 forwards 309 the payment request via a user interface on the healthcare provider device 203 to the healthcare provider. The healthcare service application 217 further transfers the specified amount of the healthcare cryptocurrency in the payment request from the encrypted digital wallet of the user to the encrypted digital wallet of the healthcare provider. The healthcare service application 217 also records 315 the payment transaction (e.g., the healthcare service, the healthcare provider, and the specified amount) in the blockchain 320.

The registered healthcare provider via a user interface on the healthcare provider device 203 to the healthcare service application 217 sends 312 a distribution of healthcare cryptocurrency received from the particular healthcare service to the health insurance company. The healthcare service application 217 receives the distribution and performs any necessary validation of the health service account of the healthcare provider (e.g., meeting payment proportions and costs that the healthcare provider contracted with the health insurance company) via a smart contract. The healthcare service application 217 forwards 314 the distribution via a user interface on the health insurance company device 204 to the health insurance company, which may confirm the distribution. The healthcare service application 217 further transfers the specified amount of the healthcare cryptocurrency in the distribution from the encrypted digital wallet of the healthcare provider to the encrypted digital wallet of the health insurance company. The healthcare service application 217 may also record 315 the distribution (e.g., the healthcare service, the healthcare provider, the health insurance company, and the specified amount) in the blockchain 320.

### Example Healthcare Transactions

FIG. 3B is another example method and system 350 for performing a healthcare transactions in embodiments of the present invention. In FIG. 3B, a consumer generates and signs 342 a payment transaction (transaction #1 341) for a healthcare service provided by a healthcare provider in an agreed upon amount using the consumer's public key. The consumer via a user interface on a consumer device 202 to the healthcare service application 217 sends 351 over network 335 the payment transaction 341. The healthcare service application 217 forwards 353 the payment transaction 341 to the health care provider via a user interface to the healthcare provider device 203. The healthcare provider confirms the payment transaction 341 by signing the payment transaction 341 with the healthcare provider's private key 343. In response to the healthcare provider signing the payment transaction 341, the healthcare service application 217 transfers the payment amount from the digital wallet of the consumer to the digital wallet of the healthcare provider. The healthcare service application 217 also broadcasts 354 over network 246 the payment transaction 341 (with only the healthcare service and payment amount and leaving out any confidential patient information). The broadcasted payment transaction 341 is recorded in the blockchain 320.

The healthcare provider then generates and signs (using the healthcare provider's public key) distribution transactions (e.g., transaction #2a 355, transaction #2b 356, and transaction #2c 357) to health insurance companies in accordance with the distribution percentages in contracts the healthcare provider entered with the health insurance companies. To confirm the distribution transactions, the health insurance companies sign the transaction with their respective private keys. Based on the confirmations by the health insurance companies, the healthcare service application 217 (via smart contracts 345), divides the payment amount into the respective distribution percentages, and deposits the divided payment amounts into the individual digital wallets of each of the health insurance companies. The healthcare service application 217 also broadcasts over network 246 the distribution transactions 355, 356, 357 (with only the health insurance company receiving the distribution and the distribution amount). The broadcasted distribution transactions 355, 356, 357 are recorded in the blockchain 320.

### Issues in Data Storage

Medical data storage is increasing in complexity with each advancement of healthcare technology. The days of using paper and pen at medical facilities to record medical data and using rudimentary file storage are gone. With the introduction of computers and the Internet to medical data storage, the accessing of medical data has met with multiple obstacles. Designed to have faster access in a simple and organized format, electronic medical records (EMRs) are now maintained locally at each medical facility. The medical data in EMRs is locked up as invaluable information only the patient and their healthcare provider (e.g., doctor) of the medical facility can access. The healthcare systems managing the EMRs are centralized and rely heavily on the technical team of each medical facility to maintain and provide security for the medical data. That is, the healthcare systems have improved the accessibility to medical data for both the patient and healthcare provider of a medical facility, but still lack accessibility to the medical data when the patient changes healthcare providers or medical facilities (or for other uses or applications).

For example, the centralization of the system management of the EMRs leave relevant medical data for a patient isolated at a medical facility. In this way, patients transferring between healthcare providers are tasked with transferring of their own medical data. Such a transfer process is time consuming, error-prone, and susceptible to loss of data with each transfer. Hospitals and other medical facilities claim the security of patients' data is of utmost importance, yet fall short in such transitional phases. The security of the patient is jeopardized not only during the transfer process, but also when the patients' medical data is accessed during visits to the medical facility.

Embodiments of the present invention is directed to a medical data storage system that is decentralized, such that the medical data (EMRs) may be accessed across medical facilities. The medical data storage system further enables access to the stored medical data in a manner that ensures security and privacy of the data. Embodiments of the decentralized data system are implemented using blockchain technology. Further the embodiments address the issue of demands on medical data access from a data storage system as the size of such medical data increases. The embodiments recognize that not all medical data is necessary or adequate for certain types of uses and applications, and provides an improved structuring (categorizing) of the medical data that enable practical search speeds for different types of uses/applications.

The medical data storage system of these embodiments implements two types of decentralized ledgers (e.g., decentralized blockchain ledgers), a private ledger and a public ledger. The two types of ledgers create multiple access points for inputting or searching the medical data based on the use or application of the medical data. A healthcare provider (HCP) stores personal and private medical data of patients in the private ledger. To store the medical data to the private ledger, the embodiments require identification by a dual login that includes the public key of both the HCP and a patient (e.g., blockchain cryptographic public keys). The medical data is stored in the private ledger associated to the public keys of both the HCP and the patient. The stored medical data may be organized based on the medical transactions performed during each patient visit to the HCP. The stored medical data may also be associated with flags that categorize and generalize the content of the medical data. The medical data may then be de-personalized and de-privatized (e.g., reduced to unique flags that categorize the medical data in a de-personalized and de-privatized manner, cost information associated with the medical data, and such) and stored in the public ledger. From the public ledger, the de-personalized and de-privatized medical data may be shared with the public for searching based on various applications (e.g., financial, research, and such).

### Method/System for Storing/Accessing Medical Data

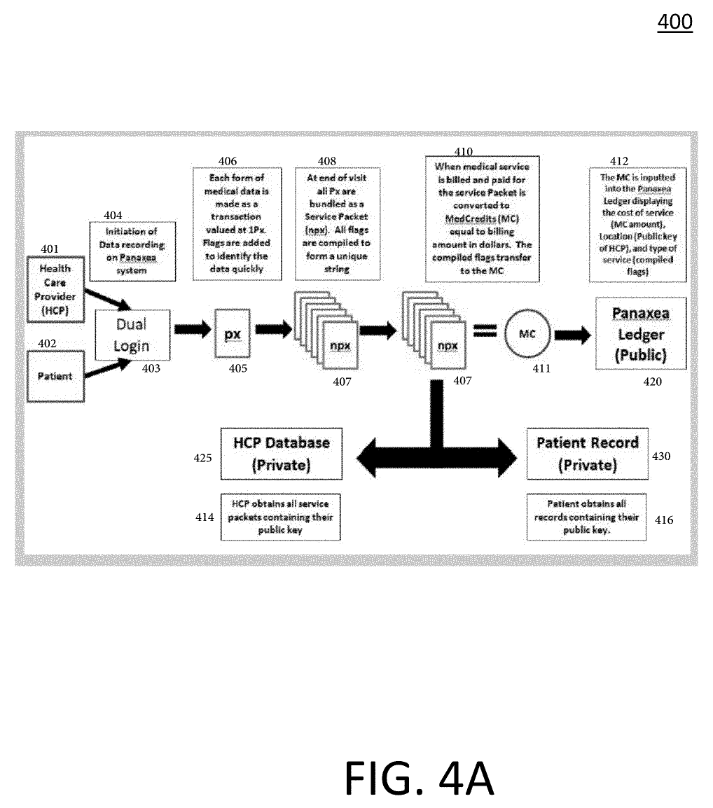

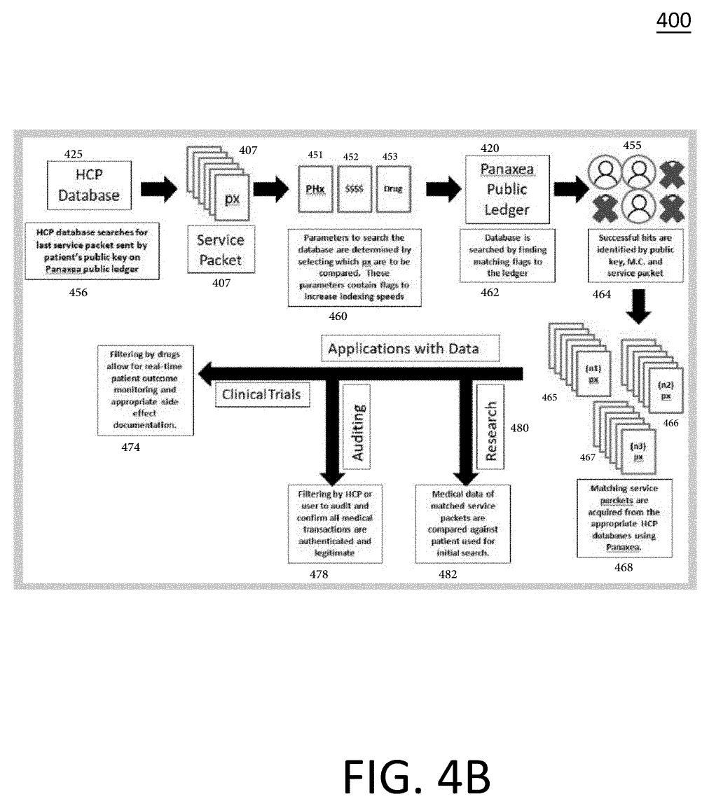

FIG. 4A is an example system (and method) 400 for storing and accessing medical data in embodiments of the present invention. The system 400 may comprise a healthcare service application processor executing a healthcare service application. A the patient user 402 and healthcare provider (HCP) 401 may access the healthcare service application through a user interface executing on a computer device and communicatively coupled to the healthcare service application.

The system (e.g., Panaxea) 400 enables a HCP 401 to subscribe (register) to the system 400 (e.g., via a user interface). Upon the HCP's subscription with the system 400, the system 400 creates and assigns a cryptographically paired public key and private key to the HCP 401. All medical processional at the HCP 401 use this same public key to perform medical transactions with patients. The system 400 includes a private ledger (HCP database) 425 associated to the HCP 401 that securely stores the EMRs of a patient user 402 that uses the HCP 401 for medical services. Each EMR contains the medical transactions (e.g., ordered tests, procedures, diagnosis, other billing expenses, and the like) 405 of a patient user 402 configured into service packets 407. Each service packet 407 contains the set of medical transactions 405 of the patient user 402 during a particular medical visit with the HCP 401. The service packet 407 also contains the public key of both the HPC 401 and the patient user 402. Through the system, the HCP has access to the HCP private ledger and medical data provided by medical professionals of the HCP.

The first time the patient user 402 visits a subscribed HCP (e.g., HCP 401), the patient user 402 registers with the system 400 (e.g., via the user interface). During registration, the system 400 creates and assigns a cryptographically paired public key and private key to the patient user 402. The patient user 402 may obtain a physical form of identification which contains both the public key and private key of the patient user 402. A patient provides personal and private information to the HCP 401 during registration (e.g., medical history, contact information, and such), which is recorded by their HPC 401 within the private ledger 425 of the HCP 401. The patient user's private key, which links to all personal and private information of the patient user 402 in the private ledger 425, is only connected with the patient user's public key at the time of creation of the key pair. The system 400 verifies both the patient user's private and public key only when the patient user 402 meets with a new HCP, which heavily reduces private information exposure and ensures all data is accurate and secure. Through the system 400, the patient user 402 has full access to their EMRs.

During a visit to the HCP, the patient user 402 may undergo various medical transactions (e.g., lab tests, medical procedures, medical diagnosis, other medical billing expenses, and the like). At step 404, the system 400 requires identification from the HCP 401 and the patient user 402 to initiate the recording of medical transactions and associated medical information at the system 400. The system 400 identifies the users 401, 402 through a dual login 403 via the user interface, where the public key of both users 401, 402 is provided to the system 400. The system 400 verifies that the provided public keys belongs to a subscribed HCP user and a registered patient user of the system 400. After the login is complete, the system 400 may transfer medical data from the private ledger of a previous HCP of the patient user 402 to the private ledger 425 of HCP 401. After dual login is complete, a log (record) is initiated that tracks the medical transactions between the HCP 401 and patient user 402. In embodiments, the system 400 provides electronic templates or forms that enable medical professionals of the HCP 401 to input medical data of the patient user 402 in a format recognizable to the medical professional.

At step 406, the HCP 401 enters at the system 400 a set of medical data (e.g., input via an electronic template) for each test, procedure, diagnosis, and other medical billing expense performed on the patient user 402 during the medical visit. The system 400 compiles in real-time a given set (form) of medical data into a medical transaction (px) 405, which is assigned a value (cost) of the transaction represented by an individual token (currency) 405, e.g., 1 Panaxea Coin or Token. The system 400 also adds flags to the medical transaction 405 to identify or classify the medical data contained in the transaction quickly. A flag is a form of shorthand coding added to transaction 405 to give basic information or a summary of the medical data contained in the transaction 405, without having to fully analyzed the transaction. Flags include identifiers that indicate what type of data the transaction 405 contains (lab test results, patient history, prescription drug list, billing information, or any other data type without limitation).

By the end of the visit, the system 400 has compiled multiple medical transactions 405, each having flags and an assigned token. Each medical transaction 405 containing a unique set of medical data relevant to the visit. At step 408, the system 400 then packages (bundles) together the multiple medical transactions 405 into a service packet (npx) 407. The service packet 407 is a complete record of the patient's medical visit, representing all the sets of medical data collected from the patient user 402 during the visit. The system 400 assigns the service packet 407 a value (cost) that is the combination of the tokens from each of the multiple medical transactions 405 forming the service packet 407, e.g., multiple Panaxea Coins or Tokens. To easily classify and recognize data in the service packet 407, the system 400 adds a unique string of code (cumulative flag) to identify or classify the service packet 407. The system 400 creates the cumulative flag by compiling the flags from each of the multiple medical transactions 405 forming the service packet 407. The cumulative flag enables quicker (streamlined) indexing and searching of service packets 407 by providing basic information, a category, or a summary of the medical data contained in the service packet 407, without having to fully analyze the medical transactions 405 of the service packet 407. The cumulative flag further enables searching the medical data of the patient user 402 without risking patient security or privacy.

The system 400 also includes the public key of both the HCP 401 and patient user 402 in the service packet 407. The private key of the patient user 402 is not included in service packet 407, so that private medical data of the patient user 401 is not accessible through the service packet 407 to ensure patient safety and digital protection. At step 430, the system 400 stores the service packet (private patient record) 407 at the private ledger (HCP Database) 425 for HCP 401 (in an EMR for the patient user 402). At step 414, the system 400 obtains and records all service packets 407 containing the public key of the HCP 401 into the private ledger 425 for HCP 401. At step 416, through the system 400, the patient user 402 can access (obtain) all records (service packets 407) of the HCP 401 containing the user's public key from the private ledger 425. If the patient user 402 visits a new HCP, the system 400 may allow the service packets 407 of the patient user 402 stored in the private ledger 425 of HCP 401 to be accessed by the new HCP by verify a dual login that specifies the public key of the patient user 402 and the public key of the new HCP. The system 400 may allow the access to the service packets 407 by transferring the service packets to the private ledger (HCP database) of the new HCP (and updating the service packets 407 to contain the public key of the new HCP). Through the system 400, the medical professionals of the HCP 401 can also access the service packets 407 of the HCP 401 from the private ledger 425 of the HCP 401 using the public key of the HPC 401.

At step 410, when the medical visit is billed to the patient user 401, the service packet 407 of the visit is part of the communication sent from the HCP 401 to the patient user 401 (including the cost represented by the combine tokens). The system 400 also converts the service packet 407 into a public packet (mc) 411, which is assigned a cost of medical services token (currency), e.g., MedCredits. The cost of medical services token does not contain a monetary value, but does reflect the cost of the medical services represented in the service packet 407 as a medical credit. The cost of medical services token (e.g., MedCredits), for simplicity, is shown in USD (1MC=1USD) in FIG. 4A. In converting the service packet 407 into a public packet 411, the system 400 compiles together the flags of each medical transaction 405 in the service packet 407 to form a unique singular flag for the public packet 411. The system 400 formats the public packet 411 to remove the personal and private information of the patient user 402. The public packet 411 only contains one or more of the location (public keys of the HCP 401 and patient user 402), the combined amount of the tokens contained in the service packet 407, the cost of medical services token, and the unique singular flag (types of service) compiled for the public packet 411. The public packet 411 may also contain an identifier that securely identifies the service packet 407 in the private ledger. The system 400 also includes a public ledger 420 communicatively coupled to the private ledger (HCP database) 425. At step 412, the public packet 411 is input/stored in a public ledger 420 together with public packets for all patient users of the system 400.

The public ledger 420 provides a transparent medical service record of all patient users of the system 400, without any sensitive information of the patient users accessible through the record. The system 400 enables the public to access the public ledger 420 in order to use the de-personalized and de-privatized medical data for various applications (e.g., research, financial, pharmaceutical, optimization in healthcare industries, and such). The data in the public ledger can be quickly indexed and searched based on the unique assigned flags (without having the overhead of analyzing all the private information details of a patient's visit to a HCP).

The following is an example use case of method 400 of FIG. 4A.

Alice goes to see Dr. Bob, her primary care physician of a new HCP. She informs the front desk that her last primary care physician of her old HCP utilized the system 400 of the present invention and therefore she already has an account with the system 400. The new HCP is able to obtain her records from the system 400 by only asking for her public key, which is on her physical identification card that was provided to her after registering with the system 400. By initiating the dual login (her public key and the new HCP's public key), the system 400 records that Alice is now in a new location for her healthcare (e.g., the new HCP's public key interacting with Alice's public key). The old HCP's database (ledger) are private, but the system 400 provide connections between HCPs that utilize the system 400. In response to the dual login, the system 400 allows and performs the transfer of Alice's EMR to the private database (ledger) of her new HCP. Through the system 400, Alice has access to her own medical record (in both the old and new HCP databases) at all times, but is unable to give access to anyone. Only by initiating the dual login can the system 400 verify, secure, and transfer her EMR of sensitive records from the old HCP to the new HCP.

### Method/System for Searching Medical Data

FIG. 4B is an example system and method for searching medical data in embodiments of the present invention. A user (e.g., clinician at a subscribed HCP utilizing the system 400) may want to understand a possible trend in a patient's health. At step 456, through the system 400, the user (via the database of the HCP 425) searches for and locates the last public packet of a service packet 407 generated by the patient's public key on the public ledger 420. By searching the last service packet 407 of this patient, the user can highlight multiple medical transactions that contain information which the user would like to analyze across the entire public ledger 420. At step 460, through the system 400, the patient user 401 determines parameters to search in the public ledger 420 based on the information in the highlighted multiple medical transactions that the user would like to analyze. The determined parameters comprise flags 451, 452, 453 (subset of flags assigned to the public packets in step 410 of FIG. 4A) to increase speed in searching across the public ledger 420.

At step 462, the system 400 searches the public ledger 420 to find flags in the patient's records (public packets) that match the flags of the selected search parameters. At step 464, the system 400 identifies successful hits (matches) 455 of public packets including the public key of the patient user 401, cost of service token, and identifiers of corresponding service packets 465, 466, 467. At step 468, the system 400 acquires the matching public packets from the appropriate HCP databases of the private ledger 420. The system 400 returns to the user the multiple data sets (medical transactions) from the matching service packet for analysis. If the user was not subscribed to the system 400, the system 400 may only return the cost of service token, amount of medical transactions, and flags of the identified service packets.

### Downstream Applications of Data

The major flaw in all existing EMR systems is the lack of flexibility with medical data. Existing centralized data storage systems lock all medical data to protect the data. This rudimentary centralized system works for its main goal, privacy, but disallows exchange of relevant data for necessary studies and audits. The system 400 of the present invention provides the balance of exchanged data encoded to protect patient confidentiality. The system 400 has dual ledgers (public and private), which allows for an indirect communication of a user's private medical data to other users, which can then be freely utilized by the other users to allow for numerous downstream applications.

In research (step 482 of FIG. 4B), obtaining service packets of similar patient groups provides an optimal tool for increasing research power for any study. For example, medical data of matched service packets may be compared against data of the patient (or other patients) used for initial research of the study. Retrospective, prospective, and even real-time studies can be performed independent of limiting variables such as distance or time of the patients. Such performance of studies provides researchers more time to study the effects of disease and relevant factors involved in its acceleration of ablation. More focus analytics at the foundation allows, in turn, more resources towards discovery and less resources depleted on research design and recruitment.

In auditing (step 478 of FIG. 4B), health insurance companies who subscribe to the system of the present invention have access to service packet information in order to verify and audit all medical charges imposed on their clients. For example, as part of an audit, a subscribed HCP may confirm that all medical transactions are authenticated and legitimate in cost (without needing to analyze the content of the medical data) by using the cumulative tokens of the returned service packets. Without the need for constant redundant claims, the process for verifying the medical charges are clean and transparent. By fundamentally changing the negotiation claims, the system facilitates a huge cut in overhead for insurance companies, which can lead to more cost-effective healthcare plans for patients.

In clinical trials (step 474 of FIG. 4B), industrial companies, such as pharmaceutical companies who are registered users of the system, can, in real-time, track all their drugs in clinic trials, from phase 1 to 4, in all patients by simply tracking all cumulative flags of the returned service packets assigned to their IP (drugs). Filtering by the drugs allows for real-time patient outcome monitoring and appropriate side effect documentation. Notification of emerging side effects are easily detected and collected allowing for quicker responses and clearances of drugs by the FDA.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A computer-implemented method for executing healthcare transactions, the method comprises:
registering a user and a healthcare provider with a healthcare service application communicatively coupled to a blockchain, the registering creates a respective healthcare service account for each of the user and the healthcare provider with the healthcare service application;
depositing, by the healthcare service application, a sum of healthcare cryptocurrency to the healthcare service account of the user;
receiving by the user from a healthcare provider, through a user interface to the healthcare service application, a payment request for a given health related service in a specified amount of the healthcare cryptocurrency;
transmitting by the user to the healthcare provider, through the user interface to the healthcare service application, a confirmation of the payment request in the specified amount;
transferring, by the healthcare service application, the specified amount from the healthcare service account of the user to the healthcare service account of the healthcare provider; and
recording, by the healthcare service application, the given health related service, the healthcare provider, and the specified amount in the blockchain.

2. The method of claim 1, wherein the healthcare cryptocurrency is configured into a two tier structure, such that:
a first tier of the healthcare cryptocurrency is purchased directly from the healthcare service application and exchangeable in two-way transactions among the user, healthcare provider, and one or more health insurance companies;
a second tier of the healthcare cryptocurrency is exchangeable in one-way transactions limited to:
the user purchasing, via the healthcare service application, the second tier healthcare cryptocurrency from one or more health insurance companies,
the user paying for a given health related service using the second tier healthcare cryptocurrency, and
the healthcare provider distributing the second tier healthcare cryptocurrency to the one or more health insurance companies at an agreed cost; and
the first tier healthcare cryptocurrency is generated into the second tier cryptocurrency based on certain conditions.

3. The method of claim 2, wherein the certain condition includes at least one of: (i) acquiring a certain amount of the first tier healthcare cryptocurrency in a healthcare service account, (ii) having the first tier healthcare currency in the healthcare service account for a particular time, and (iii) frequency of transactions using the first tier healthcare currency.

4. The method of claim 1, further comprising:
registering one or more health insurance companies with the healthcare service application, the registering creates a respective healthcare service account for each of the one or more health insurance companies;
configuring the healthcare service application to distribute healthcare cryptocurrency received in the healthcare service account of the healthcare provider to each of the one or more health insurance companies in particular proportions; and
distributing, by the healthcare service application, the specified amount transferred from the user's healthcare service account to the respective healthcare service accounts of the one or more healthcare insurance companies according to the particular proportions.

5. The method of claim 4, wherein the configured associated proportions for distributing the healthcare cryptocurrency is based on purchase agreements between the healthcare provider and the one or more health insurance companies.

6. The method of claim 1, wherein the depositing is in response to the user transmitting, through the user interface to the healthcare service application, a request to purchase the sum of healthcare cryptocurrency.

7. The method of claim 1, wherein the depositing is based on participation of the user in a subscription plan with a health insurance company, such that the health insurance company:
at least one of: (i) purchases, through an interface to the healthcare service application, a bulk amount of the healthcare cryptocurrency from the healthcare service application, and (ii) receives, through the interface to the healthcare service application, a distribution of the healthcare cryptocurrency from one or more healthcare providers;
stores the purchased or received healthcare currency in a healthcare service account for the health insurance company; and
periodically transfers, via the healthcare service application, a specific quantity of the bulk amount from the healthcare service account of the health insurance company to the healthcare service account of the user.

8. The method of claim 1, wherein an encrypted digital wallet is coupled to each healthcare service account for storing and processing the healthcare cryptocurrency.

9. The method of claim 1, wherein smart contracts are executed by the healthcare service application to perform transferring of the healthcare cryptocurrency and recording in the blockchain.

10. The method of claim 1, wherein a purchase of healthcare cryptocurrency is paid for using at least one of: other digital currency, U.S. currency, foreign government currency, and fiat currency.

11. A computer system for executing healthcare transactions, the system comprises:
a first computing device coupled to a user, the first computing device providing a first user interface to a healthcare service application, and a second computing device coupled to healthcare provider, the second computing device providing a second user interface to the healthcare service application; and
a healthcare service processor coupled to the first and second computing device, and configured to implementing the healthcare service application, the healthcare service application processor communicatively coupled to the blockchain, the healthcare service application processor configured to:
register the user and the healthcare provider with a healthcare service application communicatively coupled to a blockchain, the registering creates a respective healthcare service account for each of the user and the healthcare provider with the healthcare service application;
deposit, by the healthcare service application, a sum of healthcare cryptocurrency to the healthcare service account of the user;
receive by the user from a healthcare provider, through the first user interface to the healthcare service application, a payment request for a given health related service in a specified amount of the healthcare cryptocurrency;
transmit by the user to the healthcare provider, through the first user interface to the healthcare service application, a confirmation of the payment request in the specified amount;
transfer, by the healthcare service application, the specified amount from the healthcare service account of the user to the healthcare service account of the healthcare provider; and
record, by the healthcare service application, the given health related service, the healthcare provider, and the specified amount in the blockchain.

12. The system of claim 11, wherein the healthcare cryptocurrency is configured into a two tier structure, such that:
a first tier of the healthcare cryptocurrency is purchased directly from the healthcare service application and exchangeable in two-way transactions among the user, healthcare provider, and one or more health insurance companies;
a second tier of the healthcare cryptocurrency is exchangeable in one-way transactions limited to:
the user purchasing, via the healthcare service application, the second tier healthcare cryptocurrency from one or more health insurance companies,
the user paying for a given health related service using the second tier healthcare cryptocurrency, and
the healthcare provider distributing the second tier healthcare cryptocurrency to the one or more health insurance companies at an agreed cost; and
the first tier healthcare cryptocurrency is generated into the second tier cryptocurrency based on certain conditions.

13. The system of claim 12, wherein the certain condition includes at least one of: (i) acquiring a certain amount of the first tier healthcare cryptocurrency in a healthcare service account, (ii) having the first tier healthcare currency in the healthcare service account for a particular time, and (iii) frequency of transactions using the first tier healthcare currency.

14. The system of claim 11, further comprising:
one or more computing device coupled to one or more health insurance companies; and
the healthcare service processor communicatively coupled to the one or more computer devices, the healthcare service processor further configured to:
register one or more health insurance companies with the healthcare service application, the registering creates a respective healthcare service account for each of the one or more health insurance companies;
configure the healthcare service application to distribute healthcare cryptocurrency received in the healthcare service account of the healthcare provider to each of the one or more health insurance companies in particular proportions; and
distribute, by the healthcare service application, the specified amount transferred from the user's healthcare service account to the respective healthcare service accounts of the one or more healthcare insurance companies according to the particular proportions.

15. The system of claim 14, wherein the configured associated proportions for distributing the healthcare cryptocurrency is based on purchase agreements between the healthcare provider and the one or more health insurance companies.

16. The system of claim 11, wherein the depositing is in response to the user transmitting, through the first user interface to the healthcare service application, a request to purchase the sum of healthcare cryptocurrency.

17. The system of claim 11, wherein the depositing is based on participation of the user in a subscription plan with a health insurance company, such that the health insurance company:
at least one of: (i) purchases, through a third interface to the healthcare service application, a bulk amount of the healthcare cryptocurrency from the healthcare service application, and (ii) receives, through the third interface to the healthcare service application, a distribution of the healthcare cryptocurrency from one or more healthcare providers;
stores the purchased or received healthcare currency in a healthcare service account for the health insurance company; and
periodically transfers, via the healthcare service application, a specific quantity of the bulk amount from the healthcare service account of the health insurance company to the healthcare service account of the user.

18. The system of claim 11, wherein an encrypted digital wallet is coupled to each healthcare service account for storing and processing the healthcare cryptocurrency.

19. The system of claim 11, wherein smart contracts are executed by the healthcare service application to perform transferring of the healthcare cryptocurrency and recording in the blockchain.

20. The system of claim 11, wherein a purchase of healthcare cryptocurrency is paid for using at least one of: other digital currency, U.S. currency, foreign government currency, and fiat currency.

21-36. (canceled)
